(19) United States
(12) Patent Application Publication
(10) Pub. No.: US 2007/0219503 A1
(43) Pub. Date: Sep. 20, 2007

(54) RFID enabled plunger

(75) Inventors: Robert Loop, Sherborn, MA (US); Joe Peterson, Westlake Village, CA (US)

Correspondence Address: Robert Loop, 131 Bogastow Brook Road, Sherborn, MA, 01770, US

(73) Assignee: Robert Loop

(21) Appl. No.: 11/378,878

(22) Filed: Mar. 17, 2006

(51) Int. Cl. A61M 5/00

(52) U.S. Cl. 604/187

(57) ABSTRACT

A plunger in a syringe assembly for tracking flows and monitoring movements of contents in the syringe assembly includes a signal interfacing device, such as an antennae, and an electronic information storage device, such as an RFID chip, attached. The information storage device is provided to store information of the contents.

### BACKGROUND OF THE INVENTION

The present invention relates generally to a plunger for a syringe and more particularly, to plunger for a syringe assembly with a signal interfacing device such as an antenna and an electronic information storage device such as a radio frequency identification device (RFID) chip attached.

In a February 2004 Federal Drug Administration (FDA) report entitled “Combating Counterfeit Drug,” the FDA outlined its approach to assuring that Americans are protected from counterfeit drugs. It includes the critical element of implementing new technologies to better protect drug supply. The FDA report stated:
“Because the capabilities of counterfeiters continue to evolve rapidly, there is no single “magic bullet” technology that provides any long-term assurance of drug security. However, a combination of rapidly improving “track and trace” technologies and product authentication technologies should provide a much greater level of security for drug products in the years ahead. Similar anti-counterfeiting technologies are being used in other industries, and FDA intends to facilitate their rapid development and use to keep drugs secure against counterfeits.”

The FDA report further stated:
“Radiofrequency Identification (RFID) tagging of products by manufacturers, wholesalers, and retailers appears to be the most promising approach to reliable product tracking and tracing.”

### SUMMARY OF THE INVENTION

In accordance with an aspect of the present invention, a syringe assembly comprises a barrel; a plunger rod assembly inside the barrel; a signal interfacing device attached to the plunger rod assembly to transmit information; and an electronic information storage device attached to the plunger rod assembly containing information transmitted through the signal interfacing device.

### BRIEF DESCRIPTION OF THE DRAWINGS

FIG. 1 is a side, diagrammatical view of a syringe with an electronic information storage device and a signal interfacing device attached to a plunger rod assembly according to one embodiment of the present invention.

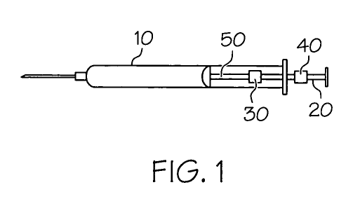

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Broadly, the present invention is a solution to address the potential FDA mandate above by attaching a signal interfacing device and an electronic information storage device to a plunger of a syringe to transmit information for human interaction for inventory control, content control, expiration control, track and tracing, and security features.

The present invention provides a syringe 10 that includes a signal interfacing device 40 and an electronic information storage device 30 affixed to a plunger rod assembly 20 to transmit information for human interaction. Information stored in such a syringe 10 may be used for inventory control, content control, expiration control, track and tracing, and security features. The signal interfacing device 40 may identify the movement of the syringe 10 through the supply chain from manufacturing to intermediate suppliers to final users by a location reading whenever the syringe 10 is transported. The signal interfacing device 40 may send out radio frequency signals containing information of the material in the syringe 10 and dispensing date to allow a receiving station to verify if the content removed is authorized to be removed or to confirm that an authorized person is intended to receive the content being removed from the syringe.

A method of attaching the electronic information storage device 30 to the plunger rod assembly 20 can be simply bonding the information storage device 30 to the surface of a plunger rod 50 through an adhesive material at an appropriate location on the surface of the plunger rod 50. It might be beneficial to manufacture a plunger rod assembly 20 with the electronic information storage device 30 embedded inside the plunger rod 50 itself to have the electronic information storage device 30 become an integral part of the plunger rod assembly 20. The process of embedding has the additional benefit of security and quality protection during the usage of the plunger rod assembly 20 in the syringe 10.

In one embodiment, the present invention provides attaching an RFID chip as the electronic information storage device 30. RFID is commonly used in the industry to provide information of the objects being track through a non-contact method through specific radio frequencies. This information can be passed to the user upon demand by triggering the specified frequency channel or by an autonomous function of RFID itself. It can be a passive device as in an apparatus with information upon demand. Or, it can be a proactive device to send information autonomously on a periodical base to a nearby reading device.

In another embodiment, the present invention provides attaching an antenna to a plunger rod assembly 20 of a syringe 10 as its signal interfacing device 40. The signal interfacing device 40 provides the function of interfacing signals between the information storage device 30 and a nearby reader or receiving a demand from a second nearby electronic source. The benefit of a device such as an antenna in the current invention is that it provides a non-line of sight transmission capability to a longer range. This capability enables the user of the syringe 10 to read information regarding the movements of the syringe 10 from a remote or non-line of sight position.

The information extracted by monitoring the signals from the antenna can be used to determine, for example, the content in the syringe barrel regarding its characteristics, allowable dosage, expiration date, date of manufacture, lot number and other relevant information. The antenna functions as a signal interfacing device 40 to register the movement of the syringe 10 through various locations within the supply chain from manufacturing to intermediate suppliers to final users. The antenna may also register the temperature ranges of the various locations that the syringe 10 had moved through. The method of attaching the antenna to the plunger rod 50 can be a simple adhesive bonding between the surfaces of the plunger rod 50 and the antenna. Or, the antenna can also be embedded inside the structure of the plunger rod 50 during the manufacturing processes to become an integral part of the syringe 10 for security (track and trace) and quality purpose.

In yet another embodiment, the antenna is an active device capable of broadcasting its locational information to allow the monitoring of its locations before and after the syringe 10 is moved or the content of the syringe dispensed. In such case, the antenna is known as a broadcaster.

In one other embodiment, the antenna transmits information on demand by another device, commonly known as an interrogator, to provide the characteristics and quantity of the content to a nearby receiver for further signal processing. The antenna is called a slave which functions as an interfacing station to receive and send out signal.

In yet one other embodiment, the signal interfacing device 40 receives information from outside source by an embedded signal receiver embedded in the signal interfacing device 40 to work as a signal receiver. The signal interfacing device 40 also transmits information to an outside receiver through an embedded transmitter in the signal interfacing device 40 to function as a signal transmitter. In addition, the signal interfacing device 40 may contain a set of decision making rules that is stored in an electrical circuitry which can be in a micro-electronic form embedded in the electric circuitry in the signal interfacing device 40. The addition of an electric circuitry adds intelligence to the signal interfacing device 40. allowing it to process information received.

It should be understood, of course, that the foregoing relates to exemplary embodiments of the invention and that modifications may be made without departing from the spirit and scope of the invention as set forth in the following claims.

We claim:

1. A syringe assembly, comprising:
a barrel;
a plunger rod assembly inside said barrel;
a signal interfacing device attached to said plunger rod assembly to transmit information; and
an electronic information storage device attached to said plunger rod assembly containing information transmitted through said signal interfacing device.

2. The syringe assembly of claim 1 wherein said signal interfacing device is embedded in said plunger rod assembly.

3. The syringe assembly of claim 1 wherein said electronic information storage device is embedded in said plunger rod assembly.

4. The syringe assembly of claim 1 wherein said signal interfacing device comprises an antennae.

5. The syringe assembly of claim 1 wherein said electronic information storage device comprises an RFID chip.

6. The syringe assembly of claim 1 wherein said signal interfacing device is a broadcaster.

7. The syringe assembly of claim 1 wherein said signal interfacing unit is a slave.

8. The syringe assembly of claim 1 wherein said signal interfacing unit comprises:
an electric circuitry embedded in said signal interfacing unit;
a signal transmitter embedded in said signal interfacing unit; and
a signal receiver embedded in said signal interfacing unit.
